(19) United States
(12) Patent Application Publication
(10) Pub. No.: US 2010/0312070 A1
(43) Pub. Date: Dec. 9, 2010

(54) Artificial Intelligence and Device for Diagnosis, Screening, Prevention and Treatment of Materno-Fetal Conditions

(76) Inventor: Shraga Rottem, Forest Hills, NY (US)

Correspondence Address: HUNTON & WILLIAMS/NEW YORK;INTELLECTUAL PROPERTY DEPT., 1900 K STREET, N.W., SUITE 1200, WASHINGTON, DC, 20006-1109, US

(21) Appl. No.: 10/596,195

(22) PCT Filed: Dec. 2, 2004
(86) PCT No.: PCT/US04/40423

§ 371 Date: Jun. 4, 2007

Related U.S. Application Data
(60) Provisional application No. 60/526,313, filed on Dec. 2, 2003.

(51) Int. Cl. A61B 5/00; G06N 5/02

(52) U.S. Cl. 600/300; 706/45; 706/50; 706/47; 706/52

(57) ABSTRACT

The present invention relates to a time-oriented artificial intelligence system to handle any diagnostic screening or treatment of complications or risks throughout pregnancy. A user can insert a problem or query relating to clinical case management during a pregnancy and receive case oriented output guiding the management of the case via at least one algorithm.

### CROSS-REFERENCE TO RELATED APPLICATIONS

This application is based upon U.S. Provisional Application Ser. No. 60/526,313, entitled AN ARTIFICIAL INTELLIGENCE AND DEVICE FOR DIAGNOSIS, SCREENING, PREVENTION AND TREATMENT OF MATERNO-FETAL CONDITIONS, the entirety of which is incorporated herein.

### BACKGROUND OF THE INVENTION

For a newborn, facing the outside world involves adaptations that start with the first milliliter of oxygen ventilating the lungs and continue throughout life. These adaptations involve all organs, systems, and an intricate network of independent and interdependent functions. A normal structural, functional and aesthetic status at birth is essential in order to enjoy what life offers and to deal with adverse situations adequately. An early and optimal detection of problems/complications during pregnancy and the best practice when handling of all risks during intrauterine life is beneficial for the patient, her family, the healthcare system, and society as a whole.

Pregnancy complication may be caused by a long list of thousands of conditions belonging to several classes:

a) existing risk of abnormal genetic inheritance at chromosomal level or at level of molecular genetics or at biochemical or metabolical level

Eg. Down's Syndrome, Turner Syndrome, and other thousands of conditions.

b) existing risk of fetal structural anomalies without detectable abnormal genetic pattern

Eg. Spina Bifida and other thousands of conditions.

c) Idiopatic fetal malformations and diseases

Eg. Hydrops Fetalis, Fetal Growth Retardation, Fetal Macrosomia

d) Fetal Diseases and pregnancy complications resulting form exposure to maternal diseases or to abnormal or untimely changes of the maternal/uterine physiology

Eg. Maternal diabetes causing fetal structural anomalies or fetal macrosomia

e) Fetal disease resulting from exposure to teratogenetic or other types of damaging agents

f) Sporadic Genetic Mutations

g) Other Problems

What is needed is an artificial intelligence software allowing for plotting, planning and handling all fetal, maternal and external pre-existent data and occurring date during pregnancy will improve the screening, detection, prevention and treatment of every case, thus improving the chance of the delivery of a neonate in the best condition to face life.

### SUMMARY OF THE INVENTION

The present invention relates to a time-oriented artificial intelligence system to handle any diagnostic screening or treatment of complications or risks throughout pregnancy.

A user can insert a problem or query relating to clinical case management during a pregnancy and receive case oriented output guiding the management of the case via at least one algorithm.

The invention allows detection of phenotype following an abnormal genotype.

The present invention provides an expert system for optimizing health during pregnancy comprising at least one database of pregnancy related health complications. Such a database may in fact include any number of databases, and such databases can be connected in any fashion, such as by hyperlinking. The system also comprises data representing time oriented information about any of said health complications The health complications may be classified into said data menus. The expert system can include at least one input for inputting diagnostic and/or screening data. The system may also include at least one indicator for reporting a decision as a function of the inputted diagnostic and screening data.

The system may include data menus. The data menus comprise categorically defined pregnancy related health conditions, said data menus being organized as a function of the pregnancy time period. These categorically defined pregnancy conditions can be classified in any number of ways.

An intelligent agent comprising at least one algorithmic rule adapted to apply to data inputted into the intelligent agent can be included. The rule can be designed to produce at least one decision about a pregnancy case. A decision may include scheduling at least one action to be taken with respect to the complication or detecting said complication. Actions that can be taken with respect to the complication include screening for the complication or treating the complication.

The intelligent agent may be configured to accept said inputted diagnostic and/or screening data and indicate the probability of the presence or absence of a pregnancy related health complication. It can do this using any number of rules. The application of said rules to inputted data, including diagnostic and screening data and health complication data is factored to report at least one decision indicating the likelihood of at least one potential health related pregnancy complication.

The intelligent agent may also include at least one incidence rule indicating the incidence of at least one pregnancy complication after birth as well as at least one incidence rule indicating the incidence of at least one pregnancy complication as a function of time during pregnancy. The application rule can be used to weigh the likelihood of a given syndrome. The agent may also include at least one classification rule directed toward classifying the at least one complication.

The intelligent agent may also include at least one association rule, said rule associating at least one decision derived from any of the above-described rules or the intelligent agent. The application of each of any of the rules included in the intelligent agent to inputted data is factored to report at least one decision indicating the likelihood of at least one potential health related pregnancy complication. Decisions or other data generated by applying the rule to diagnostic and screening data may be communicated back into the database such that it adds to the knowledge base accessible to the rules engine.

The expert system comprises a computer executed program for categorically classifying and accessing the inputted diagnostic and/or screening data and the database data. The system may also be configured to issue advisory report on future actions to be taken. Similarly, it could be configured to generate an alert based on inputted data.

### DETAILED DESCRIPTION OF THE INVENTION

The system and method of present invention provides an expert system for optimizing health during pregnancy or after birth comprising at least one database of pregnancy related health data, including pregnancy complications. Complications, as used herein, refers to any health related issue directly or indirectly related to a procedure (or risk of the procedure), treatment (including side effect or toxicity), illness, condition, abnormality or anomaly, or syndrome. The present invention manages information about complications related to pregnancy, and so complications comprise any issue that presents a concern with respect to the optimal health of either a fetus, a mother, or both. Thus, complications could comprise a syndrome, an anomalous event, nutrition or malnutrition, environmental factors, mutations at gene level, family history (e.g.: a history of retardation in the family), or even maintenance issues. Such a database may in fact include any number of databases, and such databases can be connected in any fashion. The system also comprises data representing time oriented information about any of said health complications The expert system can include at least one input for inputting diagnostic and screening data. The system may also include at least one indicator for reporting a decision as a function of the inputted diagnostic and screening data.

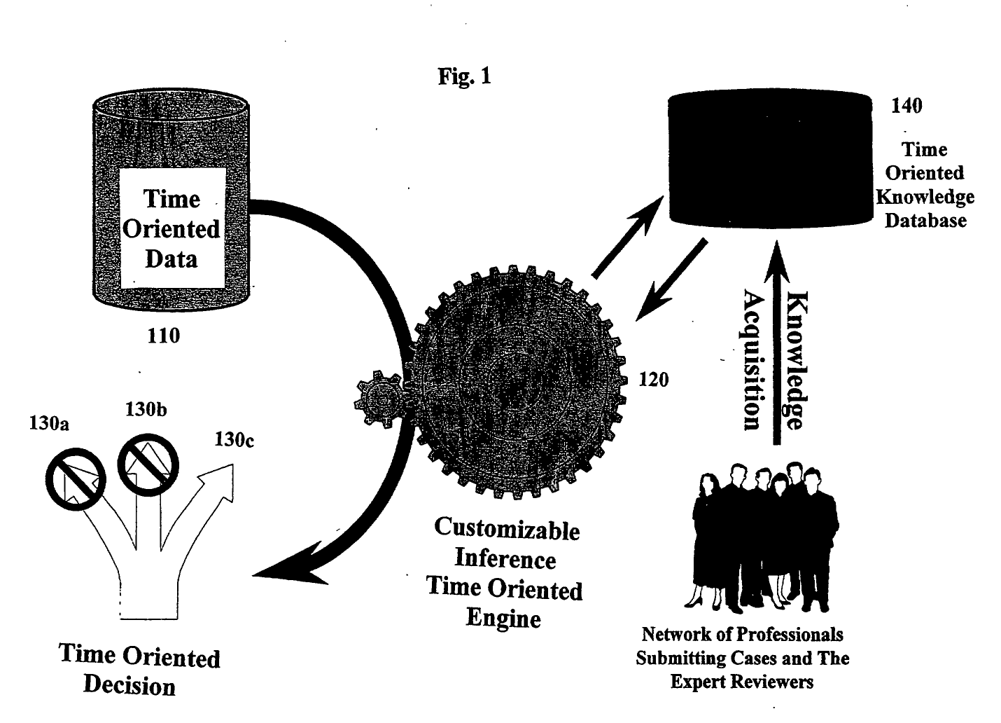

FIG. 1 shows an overall non-limiting exemplary schematic layout of the present invention. Data source 110 containing diagnostic and/or screening data from a patient is fed into an inference engine or intelligent agent 120 which outputs decisions 130a, 130b, 130c. The decisions 130a, 130b, 130c may be derived from data bases 140 using dedicated algorithms The inference engine 120 is operatively connected to at least one knowledge database 140 comprising pregnancy related time-oriented health data, neonatal related data and gene mapping. A map of the chromosomes can be linked to in the Human Genome Database (e.g:: the map of chromosome 16, http://www.gdb.org/gdbreports/Chr.16.omim.html).

The present invention may make use of, for example, a database of information related to both normal and abnormal fetal and extrauterine development over given time period. Database information may also be information enriched from the inference engine 120 itself into the database.

The knowledge data bases include time oriented menus including, inter alia, 1) genetics and genomic data base; 2) teratogen exposure before and during pregnancy; 3) maternal diseases having an impact on the fetus; and 4) events and markers related to prematurity.

The intelligent agent comprises temporal reasoning logic. Exemplary temporal logic and information about it can be found in each of the following references, the entirety of which are incorporated herein:
N. A. Lorentzos, Axiomatic Generalkation of the Relational Model to Support Valid Time Data. Proc. International Workshop on an Infrastructure for Temporal Databases, ed. R. Snodgrass, Arlington, Tex., 14-16 Jun. 1993, W1-W16.
N. A. Lorentzos, Y. G. Mitsopoulos, IXSQL: An Interval Extension to SQL. Proc. International Workshop on an Infrastructure for Temporal Databases, ed. R. Snodgrass, Arlington, Tex., 14-16 Jun. 1993, PP1-PP14.
N. A. Lorentzos, Y. Manolopoulos, Optimised Update of 2-Dimensional Interval Relations, Proc. 4th Panhellenic Conference of Greek Computer Society, Patra, 16-18 Dec. 1993, 21-33.
The Interval Extended Relational Model and its Application to Valid Time Databases. Chapter 3 in book Temporal Databases: Theory, Design and Implementation, ed. A. Tansel, J. Clifford, S. Gadia, A. Segev, R. Snodgrass, Publisher Benjamin/Cummings, USA, 1993, 67-91
C. Jensen (editor), J. Clifford, S. Gadia, F. Grandi, P. Kalua, N. Kline, N. Lorentzos, Y. Mitsopoulos, A. Montanan, S. Nair, E. Peressi, B. Pernici, E. Robertson, J. Roddick, N. Sarda, M. Scalas, A. Segev, R. Snodgrass, A. Tansel, P. Tiberio, A. Tuzhilin, G. Wuu, A Consensus Test Suite of Temporal Database Queries. Internal Report R 93-2034, Aalborg University, ISSN 0908-1216, 45 pages, November 1993, 45 pages.
C. Jensen, J. Clifford, R. Elmasri, S. Gadia, P. Hayes, S. Jajodia (editors), C. Dyreson, F. Grandi, W. Kafer, N. Kline, N. Lorentzos, Y. Mitsopoulos, A. Montanari, D. Nonen, E. Peressi, B. Pernici, J. Roddick, N. Sarda, M. Scalas, A. Segev, R. Snodgrass, M. Soo, A. Tansel, P. Tiberio, G. Wiederhold, A Concensus Glossary of Temporal Database Concepts. Internal Report R 93-2035, Aalborg University, ISSN 0908-1216, November 1993, 55 pages.
N. A. Lorentzos, DBMS Support for Time and Totally Ordered Compound Data Types. Information Systems 17(5), September 1992, 347-358.
N. A. Lorentzos, G. Mitsopoulos, Query Language for the Management of Interval Data, Proc. 1st HERMIS Conference, Athens, 25-26 Sep. 1992, 137-144.
N. A. Lorentzos, A. B. Sideridis, Valid-Time Information Systems in Agricultural Cooperatives. Proc. Agricultural Co-operatives and Development Agencies: Integrated Information Infrastructure, Athens 26-27 Nov. 1992.
N. A. Lorentzos, V. J. Kollias, The Handling of Depth and Time Intervals in Soil Information Systems. Computers and Geosciences, 15(3) 1989, 395-401.
N. A. Lorentzos, R. G. Johnson, Axtending Relational Algebra to Manipulate Temporal Data. Information Systems 13(3), 1988, 289-296.
N. A. Lorentzos, R. G. Johnson, Requirements Specification for a Temporal Extension to the Relational Model, IEEE/Data Engineering, 11(4), 1988, 26-33 (invited author).
N. A. Lorentzos, R. G. Johnson, TRA Á Model for a Temporal Relational Algebra. Proc. Temporal Aspects in Information Systems, Sophia-Antipolis, France, 13-15 May 1987 (in Temporal Aspects in Information Systems, ed. C. Rolland, F. Bodart, M. Leonard, pub. North-Holland), 1988, 203-215.
N. A. Lorentzos, R. G. Johnson, An Extension of the Relational Model to Support Generic Intervals. Proc. Int. Conf. Åxtending Database Technology, Venice, Italy, 13-15 Mar. 1988 (in Advances of Database Technology-EDBT'88 ed. J. W. Schmidt, S. Ceri, M. Missikoff, Springer-Verlag), 1988, 528-542.
R. G. Johnson, N. A. Lorentzos, Temporal Data Management. Information Update, Database Technology 1, 1987, 5-11.
N. A. Lorentzos, Y. G. Mitsopoulos, SQL Extension for Interval Data. IEEE Transactions on Knowledge and Data Engineering 9(3), 1997, 480-499.
N. A. Lorentzos, H. Darwen, Extension to SQL2 Binary Operations for Temporal Data. Invited paper, Proc. 3rd HERMIS Conference, Athens, 26-28 Sep. 1996, 462-469.
N. A. Lorentzos, Y. Manolopoulos, Functional Requirements for Historical and Interval Extensions to the Relational Model. Data and Knowledge Engineering 17, 1995, 59-86.
N. A. Lorentzos, A. Poulovassilis, C. Small, Manipulation Operations for an Interval-Extended Relational Model. Data and Knowledge Engineering 17, 1995, 1-29.
C. Vassilakis, N. A. Lorentzos, P. Georgiadis, Transaction Support in a Temporal DBMS. Proc. Int. Workshop on Temporal Databases, Zurich, 17-18 Sep. 1995 (in Recent Advances in Temporal Databases, ed. J. Clifford, A. Tuzhilin, pub. Springer), 1995, 255-271.
The SQL2 Temporal Language Design. The TSQL2 Language Committee, R. Snodgrass, I. Ahn, G. Ariav, D. Batory, J. Clifford, C. Dyreson, R. Elmasri, F. Grandi, C. Jensen, W. Kafer, N. Kline, K. Kulkarni, T. Leung, N. Lorentzos, J. Roddick, A. Segev, M. Soo, S. Sripada, ed. R. Snodgrass, pub. Klower, 1995.
N. A. Lorentzos, A. Poulovassilis, C. Small, Implementation of Update Operations for Interval Relations. Computer Journal 37(3), 1994, 163-176.
C. Jensen, J. Clifford, R. Elmasri, S. Gadia, P. Hayes, S. Jajodia (editors), C. Dyreson, F. Grandi, W. Kafer, N. Kline, N. Lorentzos, Y. Mitsopoulos, A. Montanari, D. Nonen, E. Peressi, B. Pernici, J. Roddick, N. Sarda, M. Scalas, A. Segev, R. Snodgrass, M. Soo, A. Tansel, P. Tiberio, G. Wiederhold, A Concensus Glossary of Temporal Database Concepts. SIGMOD Record, 23(1), March 1994, 52-64.
R. Snodgrass, I. Ahn, G. Ariav, D. Batory, J. Clifford, C. Dyreson, R. Elmasri, F. Grandi, C. Jensen, W. Kafer, N. Kline, K. Kulkarni, T. Leung, N. Lorentzos, J. Roddick, A. Segev, M. Soo, S. Sripada, TSQL2 Language Specification. SIGMOD Record, 23(1), March 1994, 65-96.
R. Snodgrass, I. Ahn, G. Ariav, D. Batory, J. Clifford, C. Dyreson, R. Elmasri, F. Grandi, C. Jensen, W. Kafer, N. Kline, K. Kulkarni, T. Leung, N. Lorentzos, J. Roddick, A. Segev, M. Soo, S. Sripada, A TSQL2 Tutorial. SIGMOD Record, 23(3), September 1994, 27-33.

### Temporal Databases

X. S. Wang, C. Bettini, A. Brodsky, and S. Jajodia, “Logical design for temporal databases with multiple temporal types,” ACM Trans. on Database Systems, To appear. PS file
C. Bettini, X. S. Wang, S. Jajodia, and J. Lin, “Discovering temporal relationships with multiple granularities in time sequences,” IEEE Trans. on Knowledge and Data Engineering, To appear.
C. Bettini, X. S. Wang, and S. Jajodia, “Temporal semantic assumptions and their use in database query evaluation,” IEEE Trans. on Knowledge and Data Engineering, To appear. A preliminary version appeared in Proc. ACM SIGMOD Int'l. Conf. on Management of Data, San Jose, Calif., May 1995, pages 257-268.
C. Bettini, X. S. Wang, and S. Jajodia, “Testing complex temporal relationships involving multiple granularities and its application to data mining,” Proc. 15th ACM PODS Symp., Montreal, Canada, June 1996, pages 68-78. PS file
C. Bettini, X. S. Wang, and S. Jajodia, “A general framework and reasoning models for time granularity,” Proc. 3rd Int'l. Workshop on Temporal Representation and Reasoning, Key West, Fla., May 1996. PS file
X. S. Wang, S. Jajodia, V. S. Subrahmanian, “Temporal Modules: An Approach Toward Federated Temporal Databases,” Information Sciences, Vol. 82, 1995, pages 103-128. A preliminary version appeared in Proc. ACM SIGMOD Int'l. Conf. on Management of Data, Washington, D.C., May 1993, pages 227-236.
G. Wiederhold, S. Jajodia, and W. Litwin, “Integrating temporal data in a heterogeneous environment,” in Temporal Databases, A. Tansel et al., eds., Benjamin/Cummings (1993), pages 563-579.

### Global States and Time in Distributed Systems:

P. Ammann, S. Jajodia, and P. Frankl, “Globally consistent event ordering in one-directional distributed environments,” IEEE Trans. on Parallel and Distributed Systems, Vol. 7, No. 6, June 1996, pages 665-670.
P. Ammann, S. Jajodia, and P. Mavuluri, “On-the-fly reading of entire databases,” IEEE Trans. on Knowledge and Data Engineering, Vol. 7, No. 5, 1995, pages 834-838.
P. Ammann, V. Atluri, and S. Jajodia, “The partitioned synchronization rule for planer partial orders,” IEEE Trans. on Knowledge and Data Engineering, Vol. 7, No. 5, 1995, pages 797-809.
P. Ammann, S. Jajodia, “Distributed timestamp generation in planar lattice networks,” ACM Trans. on Computer Systems, Vol. 11, No. 3, August 1993, pages 205-225.

The inference engine/intelligent agent can make decisions using cumulative weighted considerations of the following exemplary variables:
the incidence of a syndrome at birth;
the incidence or the syndrome during gestation by week of the gestation period;
the incidence of signs or markers inside each syndrome at birth;
the incidence of each sign or marker inside each syndrome by week of the gestation period;
the incidence of the associations of signs or markers at birth;
the incidence of the associations of sign or markers during gestation by week of the gestation period;
classification of each sign or marker as main, secondary, or rare with respect to a syndrome at birth;
classification of each sign or marker as main, secondary, or rare with respect to a syndrome during gestation by week of the gestation period; and
classification of each sign or marker by its natural history type (i.e. Types I-IV described herein.)

It will be recognized that the weighted value of each of the above elements and variables, as well as other variables, will vary according to case and situation, as well as accounting for other factors such as ethnicity.

According to their natural history, fetal anomalies can be classified in four types (Types I to IV): Type I—Early onset at constant gestational age; Type I—Transient condition; Type III—Variable onset or potentially unstable anomalies; and Type IV—Late onset anomalies. Examples of anomalies for Type I—Early onset at constant gestational age are: Anencephaly, bifida, Conjoined twins, Holoprosencephaly, Cyclops deformity, Osteogenesis imperfecta type II, Dextrocardia, Double collecting renal system, Anophthalmia, or Facial cleft. Examples of anomalies for Type II—Transient Conditions are: Increased NT, Pleural effusion, Pericardial effusion, Choroid plexus cysts, Hydronephrosis, Mesenteric cyst, Echogenic bowel, Oligohydramnios, Placental hypertrophy , or Cardiac Arrhythmia. Examples of anomalies for Type III—Anomalies with variable onset or potentially unstable anomalies are: Diaphragmatic hernia, Hydrocephalus, Clubfoot, Dandy-Walker, Malformation, Coarctation of aorta, Ovarian cyst, AV heart block, Exomphalos, Megacystis, or Encephalocele. Examples of anomalies for Type IV—late onset anomalies are: Agenesis of the corpus callosum, Lissencephaly, Porencephaly, Microcephaly, Intracranial arachnoid cysts, Scaphocephaly, Congenital mesoblastic nephroma, Pyloric atresia, or Osteogenesis imperfecta type IV. Additional information about natural history of fetal anomalies may be found in the following reference which is incorporated in its entirety herein: Rottem, Shraga: IRONFAN—Sonographic window into the natural history of fetal anomalies, Ultrasound Obstet. Gynecol. 5 (1995) 361-363.

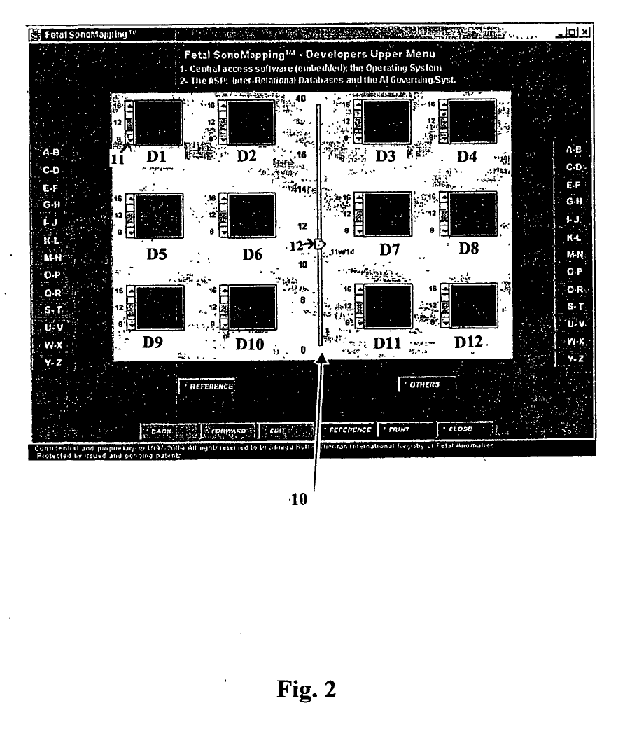

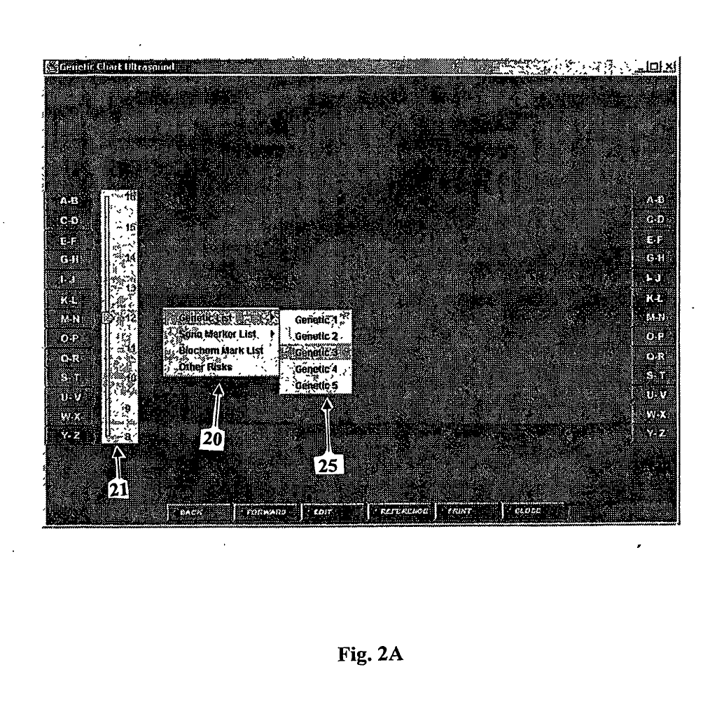

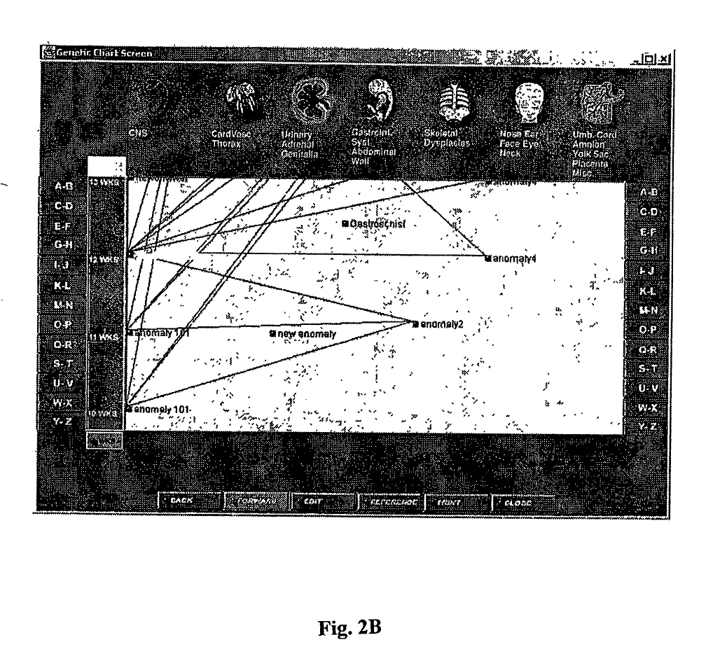

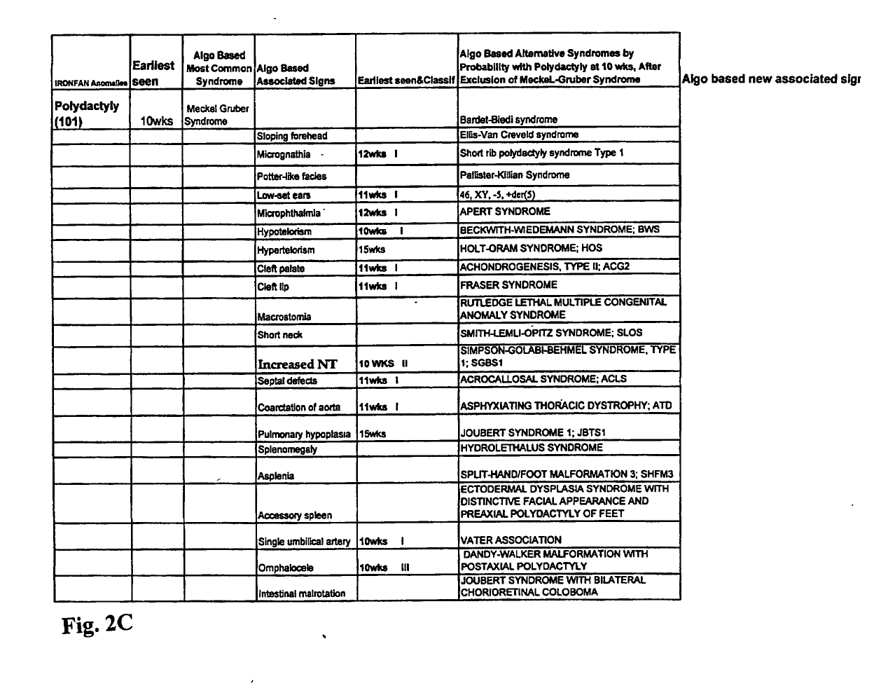

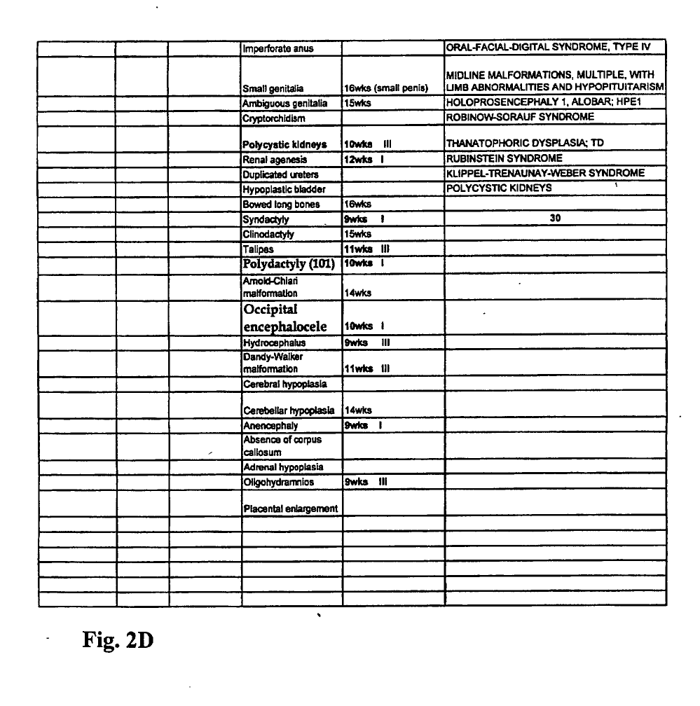

A series of non-limiting exemplary embodiments of the system and method of the invention is described in terms of display views (e.g. screen shots) in FIG. 2 to FIG. 4B. FIG. 2 shows a first screen exemplifying the relation between the time-oriented inference engine and the time-oriented knowledge bases. The time bar 10 allows coordinate access to the knowledge bases based on gestation time period. Knowledge bases D1 to D12 categorize pregnancy complications into various classes. D1 to D12 include data relating to maternal disease, fetal system development, genetic risk, risk of fetal anomaly (e.g. polydactyly) drug use before or during pregnancy or any other categorical classifications useful in managing pregnancy care. Data menus D1-D12 include data useful in diagnosing, screening, treating, and managing a pregnancy case.

The bar 10 can be designed to have normal fetal organs and organ functions or values on the left-hand side and abnormal fetal development and dysfunctions on the right-hand side, values resulting through tests such as ultrasound and other tests. A tab 12 can be designed to mark a point on a sliding scale to indicate a precise point or stage of the pregnancy, for example 11 weeks and 1 day. Each data menu D1 to D12 is designed with a timing bar including a tab scale 11. The timing scale 11 can be operatively coordinated to the time oriented bar 10 such that each data menu reflects the time of pregnancy and shows conditions related to that time of pregnancy.

FIG. 2A illustrates a screen that is presented to a user when the user chooses access data menu for example D8. The user could choose a menu for example D8 wherein health related pregnancy conditions are genetic conditions. A scale 21 is presented to indicate the time period of the pregnancy. A second data-menu 20 may show a list of options with queries which can be made relating to a genetic problem.

The options for the queries can be : a list of genetic disease; and subcategories thereof; by a marker from sonographic or biochemical investigation showing a risk or other risks.

A selection from menu 20 would open another menu 25 which provides a list of syndromes which can be detected at this gestation age. Once a syndrome is selected, the hereinabove described algorithm generates of screen as shown in FIG. 2B For example, if a genetic list were selected, the list would show chromosomal, non-chromosomal, metabolic, mendelian, and mental deficiency as the relevant complications to be screened for.

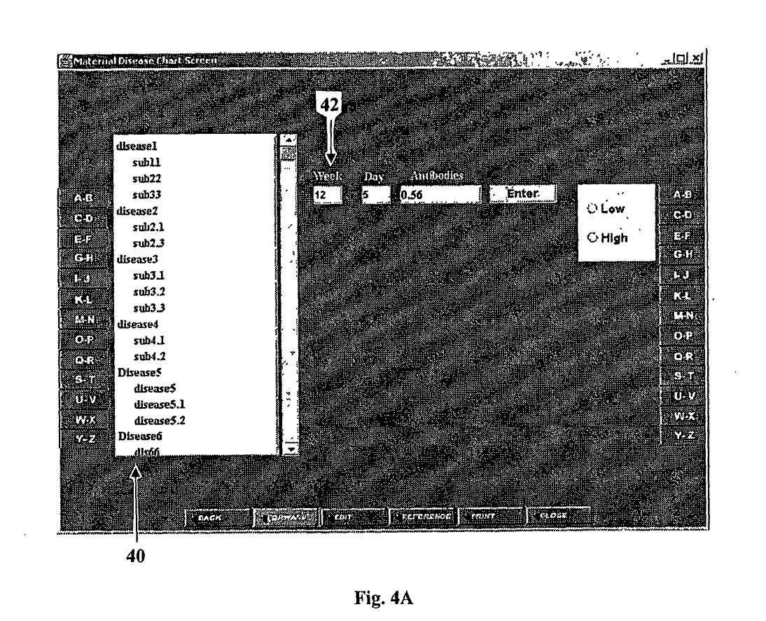

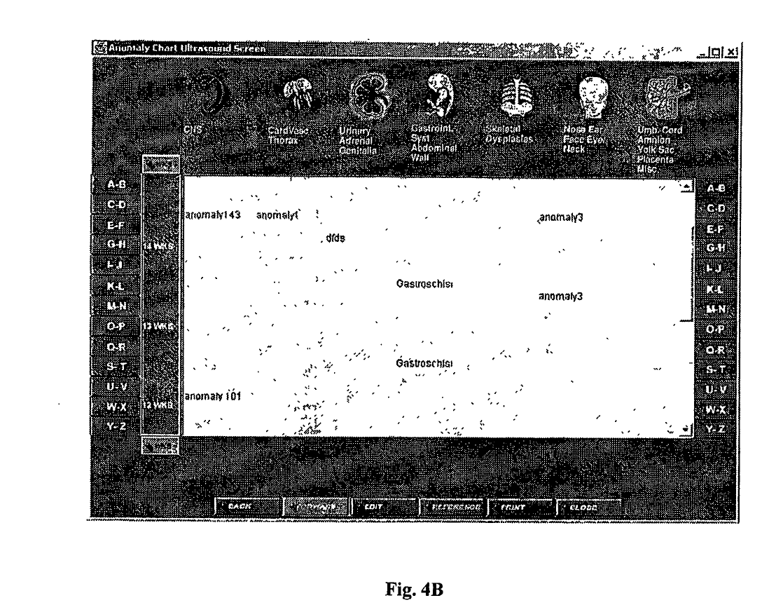

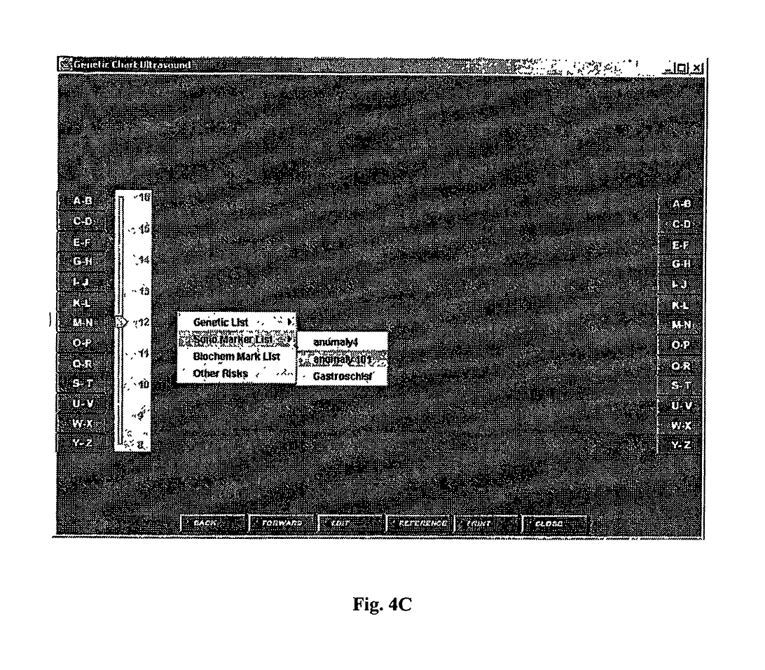

FIG. 4B is an illustration of a screen showing the anomalies and association of anomalies to look for using the algorithm based planning. The planning may change depending upon the presence or absence of an anomaly or anomalies in later scans.

FIG. 4C shows a screen in which the SonoMarker list has been selected. A query can arise form the selection of a SonoMarker from the list such as anomaly 101. In such a case the algorithm will provide the most common syndrome to look for at this time of pregnancy.

However, out of the long list of signs for a syndromes, the algorithm will provide the weighted probability of looking for the smallest number of markers to detect or exclude the syndrome. An example of such a list is shown in FIGS. 2C and 2D.

One of the urges of finding information is to look to data bases from neonatal outcomes from cases with polydactyly. This in impractical since it would involve a study of over 200 different syndromes. The algorithm from this invention directs the practitioner to the most common syndrome in the fetus with polydactyly at a particular gestational age. However, instead of looking at over 40 possible associate signs, the algorithm shown in FIGS. 2C and 2D directs the user to two signs (increased NT and Occipital Encephalocele) followed by polycystic kidneys at a later stage. According to a new evaluation of the fetus, Meckel Gruber syndrome is confirmed or excluded, the next further indicated syndrome by their probabilities and associated signs in shown.

In addition to by syndrome or by marker algorithms, the invention also includes the ability to flag a list of the least number of the markers to detect the maximum number of syndromes/diseases at a given gestational age. In addition to above-describes software, this can be achieved by a jog dial with programmable button.

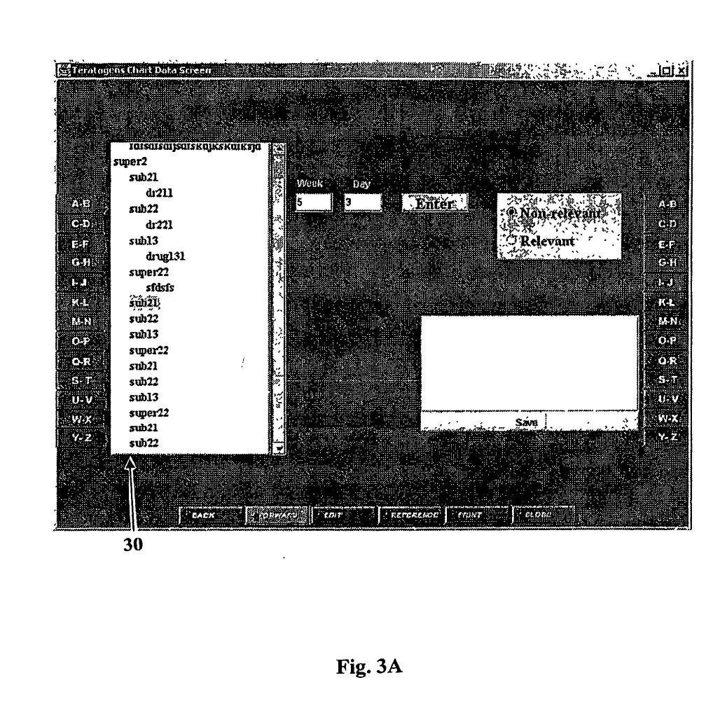

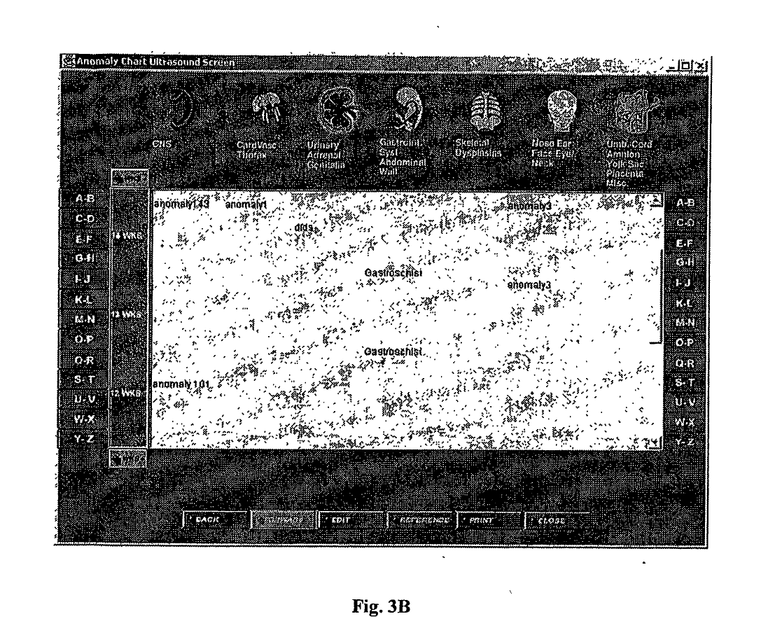

Two additional queries which can be generated using the algorithm of this invention are shown in FIG. 3A and FIG. 4A. FIG. 4A shows the query relating to possible impact from maternal disease on fetal health at a given time in the gestation.

FIG. 3A relates to the query to the inference engine and algorithm on the possible impact on the fetus from maternal exposure to a teratogen.

FIG. 3A displays one of possible embodiments of the query mode on the possible impact of maternal exposure to any teratogen at any time in gestation. The classes and subclasses are shown in menu 40. The query would include selection of a drug, time of exposure in weeks and days and dose (not shown in screen). The inference engine and algorithm will declare as non relevant or relevant to fetal health. In case of non-relevance the screen will the reassuring reasons for non-relevance. If the answer is relevant as dictated by the inference engine and algorithm the output will indicate the time for detection of possible problems in the fetus as shown in FIG. 3B.

FIG. 3A shows another example of the screen that could be accessed from the first data menus D1, D2, D3 from the access screen of FIG. 2 For example, a user could select a menu D3 of conditions related to teratogens. At FIG. 4A is shown list or menu 40 of pregnancy complications, here being teratogens, exposure to a drug for example. A display 42 shows the time of exposure to the teratogen, for example a drug taken during week 5, day 3, and dose of the drug (not shown here). A result could then report information about the drug exposure and the time of pregnancy. For example, a report box 44 could indicate whether the exposure to the teratogen is relevant to fetal development or is irrelevant. If irrelevant, the system can be configured to give a reason in second reporting box 46. A time oriented rule indicates the relationship between the diagnostic and screening information and the time of pregnancy. As an additional feature, the expert system could adapted to prompt a pregnant patient, during a sonogram for instance, and ask her if she has taken a given drug. If so, the intelligent agent process that information as described with respect to a teratogen.

If the exposure is relevant, the user can then bring up a page shown at 3B similar to that later described in FIG. 4B, with the time period being directed toward the time of pregnancy of the present example. The complications, results, actions, anomalies etc., would be related to the relevant effect of the teratogen, although it need not be limited to this.

FIG. 4A displays the possible impact on the fetus of a maternal disease during pregnancy. Menu 30 shows a list of possible maternal diseases. According to the week of gestation, the tests relating to the disease such as antibody levels and other tests, the inference engine and the algorithm will provide an output indicating whether the values of the tests of maternal disease will be too high or too low considering to what would be normal levels for the fetus. According to high or low level, a chart, shown at FIG. 4B will indicate the follow up with the fetus in the same manner as with the teratogen.

FIG. 4A shows another example of a screen that could be accessed from the first data menus D1, to D12 from the access screen of FIG. 2. For example, a user could select a menu D2 of conditions related to maternal diseases, for example anemia, diabetes, liver disease, renal failure, etc.). At FIG. 4A a display 42 for the time of pregnancy may be presented to user. For example the screen could show that a pregnancy case is on week 12, day 5. Also presented can be a list or menu 40 of pregnancy complications, each of which are associated with that time period and the selected condition of first menu D1 of FIG. 2. For example, the list could comprise diseases and subcategories of these diseases. The display could also present a user with a diagnostic and screening information, such as the level of antibodies present in the blood (e.g. 0.56). This may be data inputted from any test. The display could also show additional diagnostic and screening data, such as when the disease first began (not shown). A result could then report information about the diagnostic and screening information and the time of pregnancy. For example, the screen could indicate whether, given the disease, the amount of antibodies is low or high with respect to the effect on the fetus the 12th week and 5th day of pregnancy. A time oriented rule could be designed to indicate the relationship between the diagnostic and screening information and the time of pregnancy.

The user can then bring up a page similar to that already described in FIG. 3B, with the time period being directed toward the time of pregnancy of the present example. The complications, results, actions, anomalies etc., would be related to the maternal diseases as that was the initial menu selected, although it need not be limited to this.

Additional Features

A feature that may be included in the expert system is one where the system may be configured to issue advisory report on future actions to be taken. It could also be configured to issue an alert based on the need to take an action where such an action should have been taken earlier but was not. Similarly, it could be configured to generate an alert in the event that there was a misdiagnosis in the past. The expert system could issue such an alert when, for example, an earlier diagnosis without the benefit of the present invention diagnosed a condition or syndrome that the system knows cannot co-exist with an anomaly that was previously diagnosed as well. The system could then be adapted to indicate a new course of treatment or other actions based on the misdiagnosis.

The expert system may also comprise an operating system comprising an input for data relating to mother's condition and the fetus's condition. The data about the fetus may include the gestational age of the fetus. The gestational age can be is established by any diagnostic and screening method, including for example an ultrasonographic method, said ultrasonographic method including fetal biometer. A scaled plotting tool may also be included to plot inputted test result data, wherein the inference engine can output a decision as a function of the plotted data.

The expert system of the present invention may embedded into any diagnostic and screening device. It may also be accessible by the web to allow remote use by any user.

It should be understood that the above description is only representative of illustrative embodiments. For the convenience of the reader, the above description has focused on a limited number of representative samples of all possible embodiments, samples that teach the principles of the invention. The description has not attempted to exhaustively enumerate all possible variations or even combinations of those variations described. That alternate embodiments may not have been presented for a specific portion of the invention, or that further undescribed alternate embodiments may be available for a portion, is not to be considered a disclaimer of those alternate embodiments. One of ordinary skill will appreciate that many of those undescribed embodiments, involve differences in technology rather than differences in the application of the principles of the invention. It will be recognized that, based upon the description herein, most of the principles of the invention will be transferable to other specific technology for implementation purposes. This is particularly the case when the technology differences involve different specific hardware and/or software. Accordingly, the invention is not intended to be limited to less than the scope set forth in the following claims and equivalents.

1. An expert system for optimizing health during pregnancy comprising:
at least one database of pregnancy related health data including data representing time oriented information about pregnancy health complications;
at least one input for inputting diagnostic and screening data, including time oriented information about said diagnostic and screening data; and
at least one indicator for reporting a decision as a function of the inputted diagnostic and screening data and said pregnancy related heath data.

2. The expert system of claim 1, the system comprising:
a plurality of time oriented data menus, said data menus comprising categorically defined pregnancy related health conditions, said data menus being organized as a function of the pregnancy time period, said health complications being classified in said data menus.

3. The expert system of claim 2, wherein the categorically defined pregnancy conditions comprise:
inherited genetic abnormalities;
fetal structural anomalies without detectable abnormal genetic pattern;
idiopathic fetal malformations and diseases;
fetal disease from pathology of physiology of mother;
teratogenetic or other exposures; or
sporadic genetic mutations.

4. The expert system of claim 1 wherein said report of time oriented information comprises:
data representing the earliest known time for detection of the health complication; or
data representing a course of action regarding the complication;
data representing the likelihood of such complication; or
data representing the class of a complication.

5. The expert system of claim 1, wherein the inputted diagnostic and screening data includes data from:
a diagnostic and screening tool;
a diagnostic and screening test; or
information garnered from a person reporting on a pregnancy; or
a report on the patient.

6. The expert system of claim 5, wherein the diagnostic and screening tool comprises:
an ultrasound pattern recognition device;
a genetic testing device;
a genetic counseling system;
a device for biochemical testing; or
a magnetic resonance device.

7. The expert system of claim 5, wherein the diagnostic and screening tests comprises:
a genetic test;
an ultrasound test; or
a biochemical test.

8. The expert system of claim 5, wherein the information garnered comprises:
information from a patient interview;
information provided by someone other than patient; or
information volunteered by a patient.

9. The expert system of claim 1, said system comprising an intelligent agent further comprising at least one algorithmic rule adapted to apply to data inputted into the intelligent agent, said rule designed to produce at least one decision about a pregnancy case.

10. The expert system of claim 9, wherein said decision comprises:
scheduling at least one action to be taken with respect to the complication, said action including an action for screening for at least one said complication;
treating said complication.

11. The expert system of claim 5, wherein the report on the patient comprises:
a patient history.

12. The expert system of claim 1, wherein the input comprises:
a scaled plotting tool for plotting said inputted diagnostic and screening data, wherein
the decision is a function of the plotted data.

13. The expert system of claim 4, the expert system further comprising:
the intelligent agent, said agent being configured to accept said inputted diagnostic and screening data and indicate the probability of the presence or absence of a pregnancy related health complication.

14. The expert system of claim 1, said, system comprising a computer executed program for categorically indexing:
inputted diagnostic and screening data; and
database data
into any of said at least one said menus.

15. The system of claim 9, wherein said report indicates:
a weighted analysis as a function the intelligent agent indicating the presence, absence or probability of the presence or absence of said complication; and
a future action to be taken with respect to said weighted analysis.

16. The expert system of claim 15, wherein the future action is:
at least one screening for at least one health complication; or
at least one treatment for at least one health complication.

17. The expert system of claim 15, wherein the system is configured to issue a report advisory report on future actions to be taken.

18. The expert system of claim 1 further comprising:
an operating system comprising an input for data relating to mother's condition and the fetus's condition wherein said data about the fetus includes the gestational age of the fetus.

19. The expert system of claim 18 wherein the gestational age is established by a diagnostic and screening method.

20. The expert system of claim 19 wherein the diagnostic and screening method comprises an ultrasonographic method, said ultrasonographic method including fetal biometer.

21. The expert system of claim 1, wherein the system is embedded into a diagnostic and screening device.

22. The expert system of claim 24, wherein the diagnostic and screening device comprises any one of:
an ultrasound pattern recognition device;
a genetic testing device;
a genetic counseling system;
a device for biochemical testing; or
a magnetic resonance device.

23. The expert system of claim 1, wherein the at least one database of pregnancy health complications comprises a database comprising the human genome.

24. The expert system of claim 23, wherein the system comprising the at least one database of pregnancy health complications is operatively connected to the database comprising the human genome.

25. The expert system of claim 1, wherein the system is accessible online.

26. The expert system of claim 1, wherein the inputted diagnostic and screening data includes data inputted in response to a prompt generated by said system.

27. The expert system of claim 1, wherein the system is comprises feature extraction or reverse feature extraction.

28. The expert system of claim 9, wherein the intelligent agent comprises at least one algorithm configured to process a plurality of values including:
the incidence of a syndrome at birth;
the incidence of the syndrome during gestation by week of the gestation period;
the incidence of at least one sign or marker for each syndrome at birth;
the incidence of the at least one sign or marker during gestation by week of the gestation period;
the incidence of any association of the signs or markers at birth;
the incidence of the association of sign or markers during gestation by week of the gestation period;
classification of the at least one sign or marker as main, secondary, or rare with respect to a syndrome at birth;
classification of the at least one sign or marker as main, secondary, or rare with respect to a syndrome during gestation by week of the gestation period; or
classification of each at least one said sign or marker by at least one natural history type.

29. The expert system of claim 9, wherein the intelligent agent comprises an algorithm configured to apply to time oriented data of a plurality of signs or markers such that the maximum percentage of a plurality of syndromes associated with said signs or markers are captured from a minimum number of markers.

30. The expert system of claim 29, wherein the system is operatively connected to a jog dial selector with programmable memory, said jog dial being adapted to adjust the number of the plurality of signs or markers such that a higher maximum percentage of said plurality of syndromes are captured.
